(19) United States
(12) Patent Application Publication
(10) Pub. No.: US 2012/0258488 A1
(43) Pub. Date: Oct. 11, 2012

(54) Systems and Methods for Electrophysiological Activated Cell Sorting and Cytometry

(75) Inventors: Oscar J. Abilez, San Jose, CA (US); Frank B. Myers, Berkeley, CA (US); Luke P. Lee, Orinda, CA (US); Christopher K. Zarins, Menlo Park, CA (US)

(73) Assignees: The Regents of the University of California; The Board of Trustees of the Leland Stanford Junior University

(21) Appl. No.: 13/443,766

(22) Filed: Apr. 10, 2012

Related U.S. Application Data
(60) Provisional application No. 61/474,213, filed on Apr. 11, 2011.

(51) Int. Cl. G01N 27/02; C12M 1/42

(52) U.S. Cl. 435/34; 435/287.1

(57) ABSTRACT

Provided herein are methods and systems for non-genetic, label-free cell purification (i.e., cell cytometry and sorting), which classifies cells based on their spontaneous electrophysiological response or their electrophysiological response to a stimulus. For example, in one embodiment, there is provided a method of cell sorting comprising: stimulating a cell with a stimulus; sensing a response evoked by the cell based on the stimulus; identifying a phenotype of the cell based on the evoked response; and sorting the cell based on its phenotype. In one embodiment, the stimulus may be an electrical stimulus, a mechanical stimulus, an optical stimulus, a thermal stimulus, a chemical stimulus, or any combination thereof. The cell phenotype may be, for example, cardiomyocytes, neurons, smooth muscle cells, or pancreatic beta cells.

### CROSS-REFERENCE TO RELATED APPLICATIONS

This applications claims the benefit under 35 U.S.C. §119(e) of U.S. Provisional Patent Application No. 61/474,213, filed on Apr. 11, 2011, the entire disclosure of which is incorporated by reference herein.

### STATEMENT REGARDING FEDERALLY-SPONSORED RESEARCH AND DEVELOPMENT

This invention was made with Government support under contract No. HL089027 awarded by the National Institutes of Health (NIH). The Government has certain rights in this invention.

### BACKGROUND

Stem cell therapies hold great promise for repairing tissue damaged due to disease or injury. One of the major obstacles in translating stem cell biology into tissue replacement therapy, however, is the lack of effective purification methods that specifically isolate and separate desired cells for implantation from cells that may have adverse effects on the performance of the implanted graft or the health of the patient. Conventional cell sorting requires exogenous fluorescent labeling of cell surface markers and, for many cell types of interest (e.g., cardiomyocytes), suitable surface markers have not been identified. Furthermore, labeling molecules may pose a risk to the patient and the functionality of the graft. Genetically-modified cells, which express a fluorescent reporter gene or confer antibiotic resistance for selected survival under a cell-type-specific promoter, can also be used. But genetic modification carries a tumorigenic risk. What is needed is a high-throughput, label-free purification method that does not require genetic modification of the cells.

Electrophysiological signals are the gold standard for assessing muscle and nerve phenotype. These signals, which can be measured non-invasively and without detriment to the cell, are a useful contrast mechanism for cell cytometry and sorting. Furthermore, for basic and applied research in stem cell biology and cardiovascular disease, there is great interest in exploring the heterogeneity of electrically-active cells—both those derived from stem cells and those from diseased organs. Therefore, electrophysiological cytometry and sorting would be an asset in these fields.

### SUMMARY

Provided herein are methods and systems for cell sorting and flow cytometry. More specifically, there is provided methods and systems for non-genetic, label-free cell analysis and purification, which classifies cells based on their spontaneous electrophysiological response or their electrophysiological response to a stimulus. For example, in one embodiment, there is provided a method of cell sorting comprising: stimulating a cell; sensing a response evoked by the cell based on the stimulus; identifying a phenotype of the cell based on the evoked response; and sorting the cell based on its phenotype. In one embodiment, the stimulus may be an electrical stimulus, a mechanical stimulus, an optical stimulus, a thermal stimulus, a chemical stimulus, or any combination thereof. In another embodiment, sorting of the cells is not included as only population statistics are desired for research or diagnostic purposes. The cell phenotype may be, for example, cardiomyocytes, neurons, smooth muscle cells, or pancreatic beta cells.

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use a cell sorter and cytometry instrument in accordance with the present invention. In the drawings, like reference numbers indicate identical or functionally similar elements.

FIG. 1 is an illustration of the phenotypes of electrically excitable cells, which may be identified with the present invention, and their respective electrophysiological field potential signals.

FIG. 2 (panel A) shows a conceptual diagram of a microfluidic electrophysiological cell sorter; (B) is a photograph of a custom instrumentation amplifier PCB; (C) shows an assembled micro-device consisting of a PDMS microfluidic channel bonded to a glass slide containing Pt electrodes; and (D) is an illustration of fabricated electrodes in a flow chamber.

FIG. 3 (panel A) is a schematic diagram in accordance with one embodiment of the present invention; (B) illustrates a longitudinal cross-sectional view of a flow chamber, and a circuit model illustrating field stimulation; and (C) illustrates a transverse cross-sectional view of a flow chamber, and circuit model illustrating a depolarization current and resulting field potential.

FIG. 4 (panel A) illustrates stimulus artifact suppression through the various techniques employed herein; and (B) illustrates a technique for artifact removal.

FIG. 5 illustrates signals from spontaneously beating induced pluripotent stem cell-derived cardiomyocyte (iPSC-CM) clusters.

FIG. 6 (panel A) illustrates stimulus responses of differentiated cardiomyocytes and undifferentiated embryoid bodies, before artifact subtraction; (B) shows a close-up of an evoked field potential (FP) after stimulus artifact suppression; (C) shows spontaneous FP averaged 10× to reduce noise; (D) shows an iPSC-CM cluster positioned over one detection electrode, with the differential reference electrode on the left.

FIG. 7 is a schematic drawing of a generalized computer system used to implement the methods presented herein.

FIG. 8 illustrates components of an automated, specialized computer-controlled cell sorter system.

FIG. 9 shows various embodiments of electrophysiological cell sorting.

FIG. 10 shows cell sorting based on a generalized physiological response to stimulus.

FIG. 11 shows spontaneous field potentials recorded from cells in flow at different flow rates.

FIG. 12 shows an example of a stimulator and instrumentation amplifier developed for electrophysiological cell sorting.

FIG. 13 shows example of field potential characteristics that may be used to assess cell phenotype.

FIG. 14 shows a representative software state diagram for cell sorting.

### DETAILED DESCRIPTION

Many of the cell populations currently being explored for regenerative medicine are electrically-excitable. For example, cardiomyocytes, smooth muscle cells, and neurons, all of which are electrically excitable, are sought for cardiac, vascular, and neural tissue engineering applications, respectively. Like all animal cells, electrically-excitable cells maintain concentration gradients of certain ions across their plasma membranes through the use of active ion transport proteins. Unlike other cells, however, electrically-excitable cells also feature voltage-gated ion channels which, upon activation by sufficient transmembrane electric fields, transiently open and allow ions to flow across the membrane down these concentration gradients. These ion currents lead to a voltage signal in the resistive medium surrounding the cell (i.e., an extracellular field potential signal), which can be detected with a nearby microelectrode.

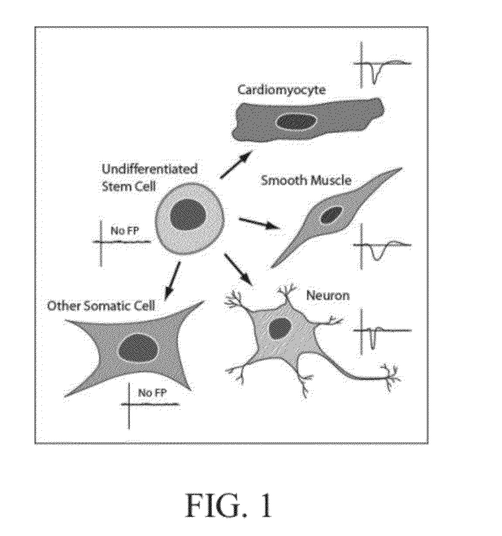

Each cell type has a characteristic protein expression pattern including many different ion channels, each with unique gating kinetics. Therefore, each cell type has a unique action potential and corresponding field potential signal (i.e., electrophysiological signature) that can provide rich phenotypic information. FIG. 1, for example, is an illustration of the phenotypes of electrically excitable cells, and their respective field potential signals. Extracellular field potential signals are unique to electrically-excitable myocytes and neural cells. Undifferentiated stem cells do not produce these signals, nor do most other somatic cell types. Furthermore, electrophysiological signals change as a cell matures from an embryonic to an adult phenotype during stem cell differentiation.

The following detailed description of the figures refers to the accompanying drawings that illustrate exemplary embodiments of a cell sorting system and methods that analyze a cell's field potential signal and electrophysiological signature. Other embodiments are possible. Modifications may be made to the embodiment described herein without departing from the spirit and scope of the present invention. Therefore, the following detailed description is not meant to be limiting.

For example, provided herein is a cell sorter system that can distinguish undifferentiated human induced pluripotent stem cell (iPSC) clusters from iPSC-derived cardiomyocyte clusters (iPSC-CM). The system utilizes a microfluidic device with integrated electrodes for electrical stimulation and recording of extracellular field potential signals from suspended cells in constant or intermittent flow. Based on automated analysis of these signals, the system directs cells into one of several outlet reservoirs. This modular microfluidic device can be parallelized to achieve throughputs relevant for research and clinical applications.

Provided herein are also non-genetic, label-free cell purification techniques, which classify cells based on their electrophysiological response to a stimulus. As many of the cell types relevant for regenerative medicine are electrically-excitable (e.g., cardiomyocytes, neurons, smooth muscle cells), these techniques are well-suited for generating highly-pure populations of desired cell phenotypes from heterogeneous stem cell progeny. As such, the cell sorting systems and techniques presented below are based on analysis of a cell's functionality rather than its physical charkteristics or surface marker expression profile. The techniques are particularly promising for purifying cardiomyocytes, which do not have reliable surface markers suitable for fluorescent labeling. The technique can also identify different subpopulations of cardiomyocytes, which would be very difficult to do with label-based strategies because label-based strategies would need to include several different labels. Label-based strategies also require different labels for the different protein channels that, together, account for the different electrophysiologic phenotypes.

Currently, there is no known way to sort cells based on their electrophysiology.

The systems and methods presented here take signals detected from suspended cells in a flow channel (or chamber), and distinguish cells using these signals; such as, for example, differentiated human iPSC-CM from undifferentiated iPSCs. Although the description below may focus on electrophysiology, a broader paradigm is envisioned wherein cell sorting is performed on a cell-by-cell or cluster-by-cluster basis, based on a cell's dynamic, functional response to a stimulus; whether the stimulus be electrical, optical, chemical, thermal, or mechanical, or any combination thereof.

In one embodiment, there is provided a method of cell sorting comprising: stimulating a cell with a stimulus; sensing a response evoked by the cell based on the stimulus; identifying a phenotype of the cell based on the evoked response; and sorting the cell based on its phenotype. In one embodiment, the stimulus may be an electrical stimulus, a mechanical stimulus, an optical stimulus, a thermal stimulus, a chemical stimulus, or any combination thereof. The cell phenotype may be, for example, cardiomyocytes, neurons, smooth muscle cells, or pancreatic beta cells.

In another embodiment, there is provided a method comprising: flowing a cell population through a flow channel; subjecting one or more individual cells to an electrical stimulus within the flow channel; sensing an electrical response evoked by the stimulated cell; obtaining an electrophysiological signature of the stimulated cell based on the evoked electrical response; and sorting the stimulated cell based on its electrophysiological signature. The cell population may be hydrodynamically, mechanically, electrically, or acoustically focused within the flow channel. The method may further include: (1) identifying a phenotype of the stimulated cell based on its electrophysiological signature; (2) identifying the stimulated cell's developmental maturity based on its electrophysiological signature; and/or (3) evaluating the stimulated cell's cellular function based on its electrophysiological signature.

Various methods of preparing the cell population are available. For example, the cell population may be prepared by enzymatically digesting the cell population into a single cell suspension. Alternatively, the cell population may be prepared by adhering the cell population onto or within a carrier. For example, the carrier may be a micro-scale polystyrene or agarose bead. Alternatively, the cell population may be prepared by aggregating the cell population into a cluster. In one embodiment, the cell population is free of any cellular labeling and/or free of any genetic modification. It is noted that the systems and techniques disclosed herein are equally applicable to individual cells, cells on carries, clusters of cells, etc.

In one embodiment, the method presented herein includes stimulating the cell with a stimulus selected from the group consisting of: an electrical stimulus, a mechanical stimulus, an optical stimulus, a thermal stimulus, a chemical stimulus, and any combination thereof. For example, in one embodiment, the method includes: applying an electrical current pulse to the cell; and sensing an extracellular electrophysiological field potential signal evoked from the cell in response to the applied electrical current pulse. The methods presented may also quantify a parameter of the electrophysiological field potential signal. The parameter may be selected from the group consisting of: an amplitude and duration of depolarization, a sustained contraction phase, a repolarization phase, refractor period, and any combination thereof.

In another embodiment, spontaneous activity associated with electrophysiology (i.e. electrophysiological signals or optical/mechanical signals arising from the electrical activity or contraction of the cells) may be used for analysis in absence of a stimulation. In such an embodiment, all of the previously described signal parameters may be quantified, as well as the rate at which spontaneous activity occurs.

In another embodiment, there is provided a system for cell sorting including: a flow chamber having a cell inlet; an impedance analyzer coupled to the flow cell and configured to detect when a cell has entered the flow chamber; and a stimulus pulse generator having two stimulation electrodes configured to create an electrical field across the flow chamber. The system further includes: a signal detector having two sensing electrodes located on an equipotential line between the stimulation electrodes, wherein the two sensing electrodes are coupled to a differential sensing amplifier configured to detect an extracellular electrophysiological field potential signal evoked from the cell in response to the electrical field across the flow chamber. A plurality of the sensing electrodes located on an equipotential line between the stimulation electrodes may also be configured along the flow channel to detect various amplitudes of the field potential at various distances from the cell. A processing unit is coupled to the signal detector and configured to identify a phenotype of a cell in the flow chamber based on the detected electrophysiological field potential signal evoked from the cell. The processing unit may be further configured to identifying the cell's developmental maturity and/or evaluate the cell's cellular function. A cell collection chamber is coupled to the flow chamber and configured to receive a cell of interest based on the cell's phenotype. Finally, a drain outlet coupled to the flow and configured to receive unwanted cells or fluid from the flow chamber.

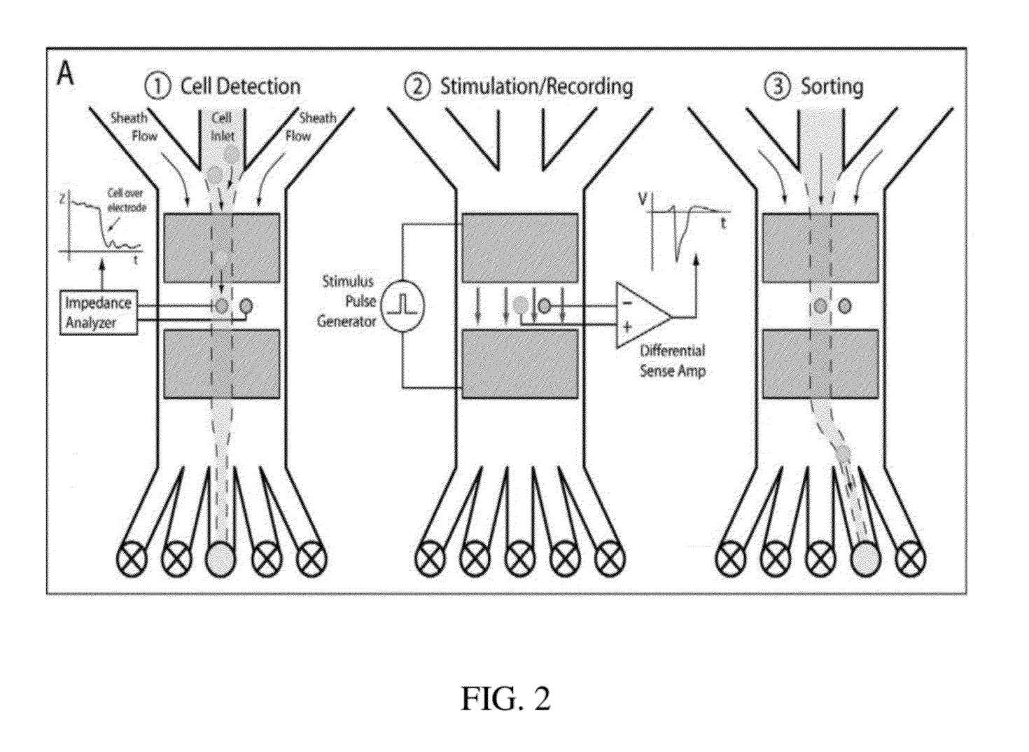

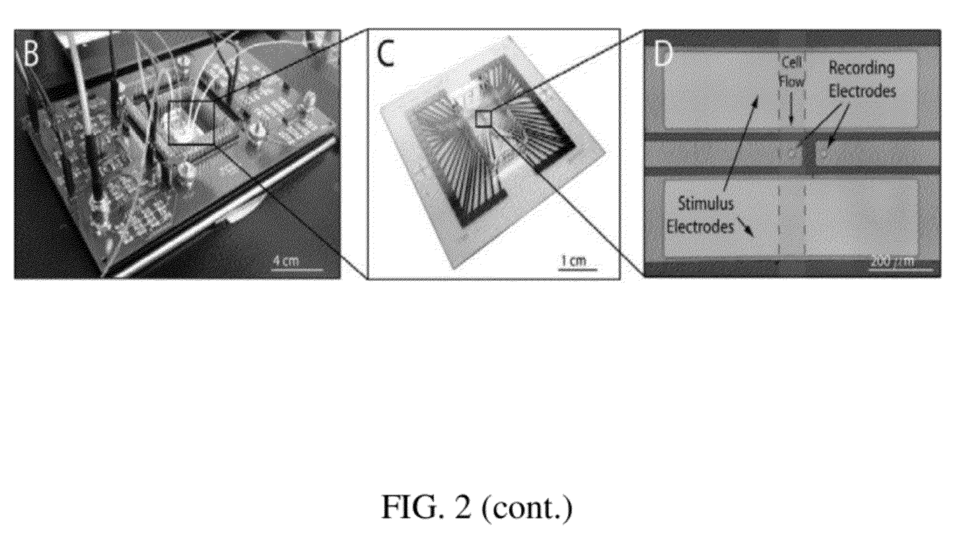

FIG. 2 (panel A) shows a conceptual diagram of a micro-fluidic electrophysiological cell sorter, in accordance with one embodiment. As shown, cells are hydrodynamically focused over detection electrodes. The presence of the cells is indicated by a drop in impedance. When the presence of a cell is detected, the flow may be stopped. Once stopped, cells are stimulated and the differential signal between the two detection electrodes is recorded. Because the detection electrodes are located on an equipotential line between the stimulus electrodes, the stimulus artifact is common mode and thus rejected. The field potential signal is then analyzed, and the cells arc sorted accordingly. FIG. 2 (panel B) is a photograph of a custom instrumentation amplifier PCB. FIG. 2 (panel C) shows an assembled micro-device consisting of a PDMS microfluidic channel bonded to a glass slide containing Pt electrodes. FIG. 2 (panel D) is an illustration of fabricated electrodes in a flow chamber.

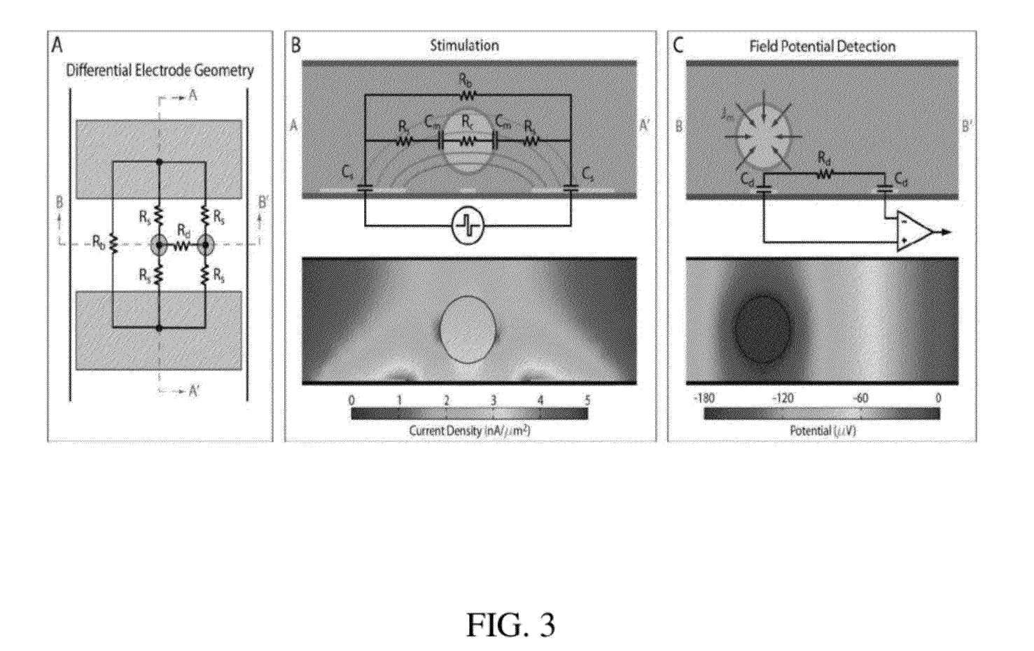

FIG. 3 (panel A) is a schematic diagram in accordance with one embodiment of the present invention. The large rectangular stimulus electrodes and small circular detection electrodes form a balanced bridge circuit, where current flows equally over each electrode (through resistances represented (Rs)). Resistance (Rb) represents the bulk resistance. Resistance (Rd) represents the resistance between the detection electrodes, which impacts signal-to-noise (SNR).

FIG. 3 (panel B) illustrates a longitudinal cross-sectional view of a flow chamber, and a circuit model illustrating field stimulation. Current is injected into the device through the double-layer capacitance Cs. A fraction of this current flows through Rs and charges up the membrane capacitance Cm. This leads to an increase in transmembrane voltage, ΔVm. If ΔVm>−30 mV, voltage-gated Na⁺ channels on the membrane open, initiating an action potential which leads to an extracellular field potential signal.

FIG. 3 (panel C) illustrates a transverse cross-sectional view of a flow chamber, and circuit model illustrating a depolarization current and resulting field potential. Excitation causes voltage-gated Na+ channels on the cell membrane to open, which allow Na+ ions to rapidly diffuse into the cell. The Na+ ion diffusion leads to a high current density and an associated ohmic voltage drop in the surrounding resistive medium, represented by Rd. This voltage can be measured by placing an electrode near the cell with a differential reference several cell radii away. The double-layer capacitance of the detection electrodes is represented by Cd.

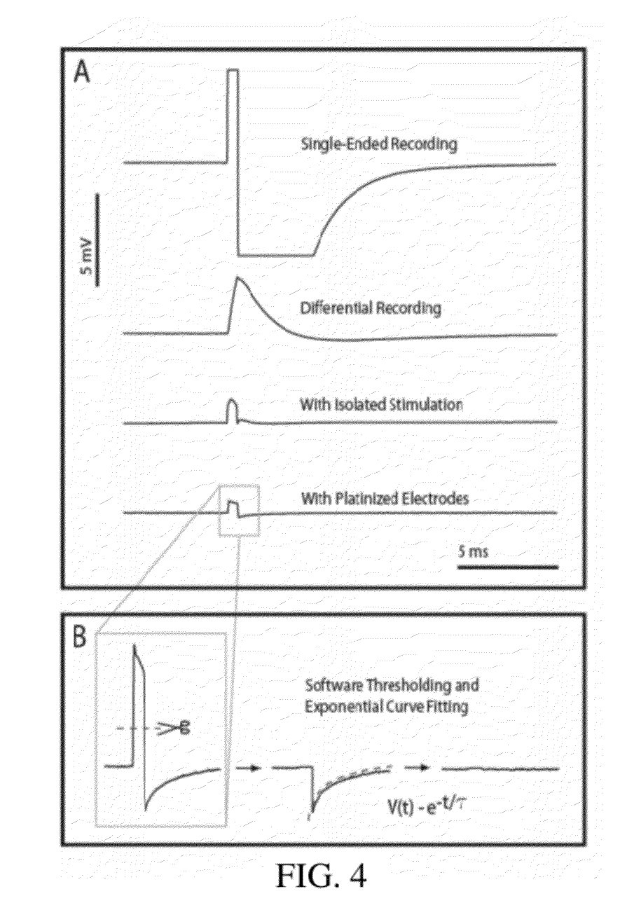

FIG. 4 (panel A) illustrates stimulus artifact suppression through the various techniques employed herein. A 100 μA, 500 us pulse was delivered in a 500 μm tall, 1000 μm wide channel via two 200 μm×1000 μm stimulation electrodes spaced 1000 μm proximal and distal to the recording electrodes. The 40 μm recording electrodes were spaced 200 μm apart. Single-ended recordings, in which one electrode was recorded with respect to a single-ended on-chip Pt reference electrode, caused dramatic amplifier saturation for 4 ms. Differential recording between the two recording electrodes dramatically reduces this artifact and eliminates amplifier saturation. When using an isolated stimulator, the recovery time drops significantly since the stimulus charge cannot discharge through the recording amplifier. Finally, platinizing electrodes helps with recovery, so we are left with a small artifact during stimulation and a subsequent RC decay, which can be removed in software. (B) Software algorithm for artifact removal.

FIG. 4 (panel B) illustrates a technique for artifact removal. The stimulus pulse is located and any samples>+100 μV are blanked, along with samples 1 ms before and 100 us after. The remaining RC decay is fitted to an exponential decay function, and this function is then subtracted from the signal.

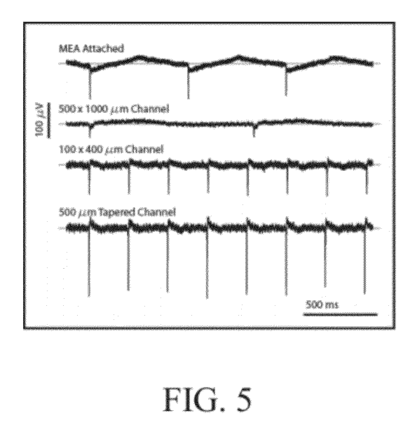

FIG. 5 illustrates signals from a spontaneously beating iPSC-CM clusters. Micro-channels enhance field potentials by confining the diffusive current density to the cross-section of the channel. Signals from spontaneously beating 200 μm iPSC-CM clusters were recorded while they were adhered on a commercial MEA, suspended in a large 500×100 μm channel, a smaller 100×400 μm channel, and a 500 μm channel in which the cells were tightly confined in a tapered region. As the ratio between channel cross-section and cluster cross-section decreased, their field potential amplitude approaches and even, in the case of the tapered channel, surpasses that seen on the MEA. This shows that detecting signals from nonattached cells in micro-channels with SNRs equivalent to those obtained with attached cells on MEAs is possible.

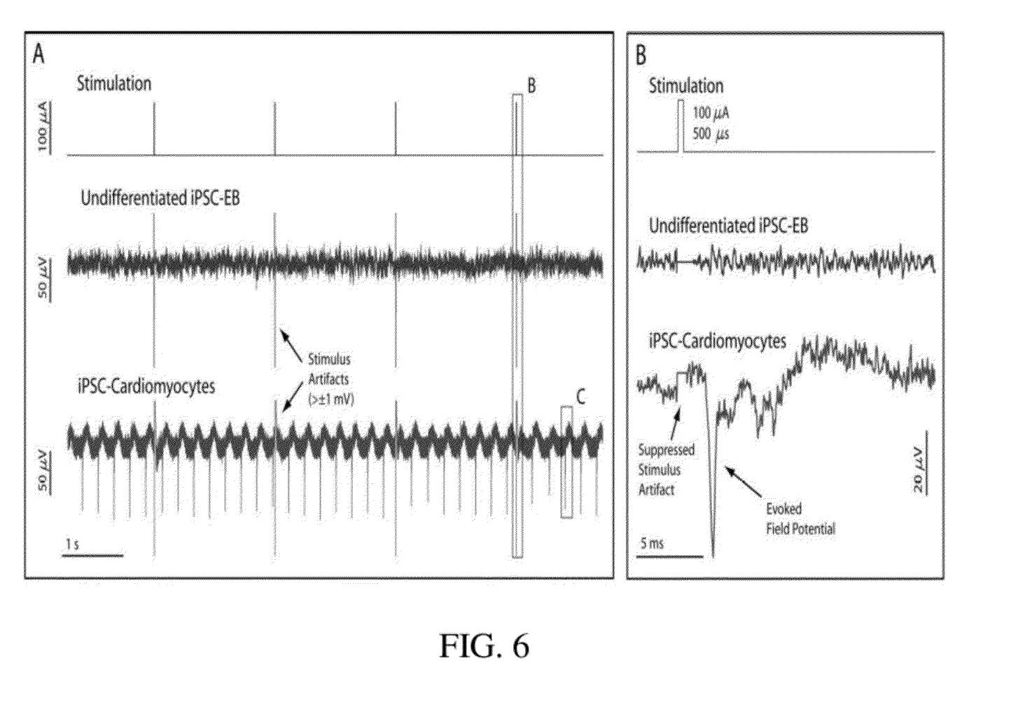

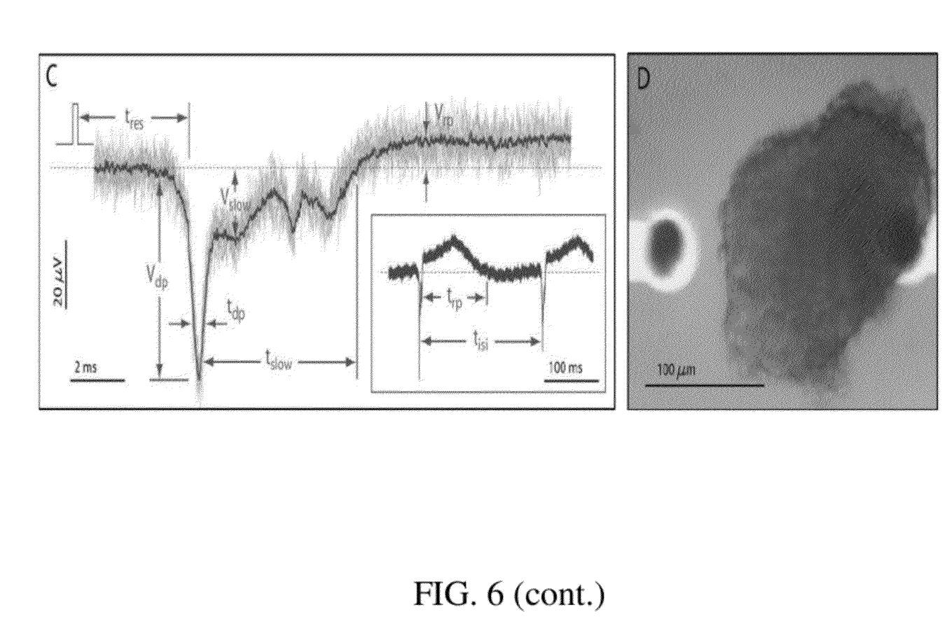

FIG. 6 (panel A) illustrates stimulus responses of differentiated iPSC-CM and undifferentiated iPSC clusters, before artifact subtraction. Cells were spontaneously beating at a rate of 5 Hz, and also responded to stimuli. Note that the first two stimuli do not result in evoked field potentials because they occurred during the refractory period. FIG. 6 (panel B) shows a close-up of an evoked field potential (FP) after stimulus artifact suppression. A −60 μV field potential is clearly visible from cardiomyocytes while undifferentiated cells produce no signal. FIG. 6 (panel C) shows spontaneous FP averaged 10× to reduce noise. Averaging allows many subtle variations in amplitude and timing parameters to be measured: response time (tᵣₑₛ), depolarization time (t_{dp}), slow current time (t_{slow}), repolarization time (tᵣₚ), interspike interval (tᵢₛᵢ), depolarization amplitude (V_{dp}), slow current amplitude (V_{slow}), and repolarization amplitude (Vᵣₚ). Inset shows two successive spontaneous FPs. FIG. 6 (panel D) shows an iPSC-CM cluster positioned over one detection electrode, with the differential reference electrode on the left. The 40 μm electrode is covered in Pt black.

To date, techniques exploring the relationship of electrophysiology to cell phenotype have been done with adherent cultures, tissue slice preparations, or in vivo. Even with cells which are adhered on sensing electrodes, field potential signals are notoriously weak. Furthermore, field stimulation produces dramatic artifacts in the recording which can obscure these signals. This is particularly problematic when stimulation and recording must occur on the same cell. The systems and methods in accordance with one or more embodiments presented herein address these problems in several ways. First, since cells are confined in a micro-channel, the ohmic voltage drop in the vicinity of the cells increases since current is confined to the cross-section of the channel. Second, a differential detection scheme is employed, placing a pair of sensing electrodes on an equipotential line in the stimulus field. This arrangement dramatically reduces the stimulus artifact seen by the sensing amplifier as compared with a single-ended recording. The spacing of the electrodes is designed to minimize thermal noise (<2 μVᵣₘₛ) and maximize the recorded field potential (50-200 μV). Third, an artifact suppression algorithm is employed, which eliminates artifact through a combination of template subtraction, linear filtering, and least squares exponential curve fitting/subtraction.

### EXAMPLES

The following paragraphs serve as example embodiments of the above-described systems. The examples provided are prophetic examples, unless explicitly stated otherwise.

### Instrumentation.

The following is a listing of instrumentation used in a sample device:

1. A custom printed circuit board (PCB) containing an instrumentation amplifier and an optoisolated, battery-powered stimulator is interfaced to the microfluidic chip via spring-loaded gold pins.
2. A glass slide coated with a thin film of indium tin oxide (ITO) is positioned underneath the device and DC current through the ITO warms the device from room temperature (˜22° C.) to 37° C. uniformly over the area of the chip.
3. Temperature on the slide is monitored using a thermistor.
4. The device is positioned under an upright microscope equipped with a video camera for visual inspection of cell positioning and contractions.
5. The entire system is enclosed in a Faraday cage to minimize power line and radio frequency (RE) interference.
6. Custom LabVIEW controller software in conjunction with a 16-bit data acquisition module (National Instruments, Austin, Tex.) is used to generate stimulus pulses and digitize signals from the device at a sampling rate of 100 kHz.
7. An LCR meter (Model 4284A, Agilent; Santa Clara, Calif.) is used to monitor the impedance between the detection electrodes, and this information is continuously relayed to the LabVIEW controller via a GPIB bus.
8. When a cell is detected, the LabVIEW controller turns off the LCR meter's interrogation signal and disconnects it from the detection electrodes via two analog switches. At that point, the stimulus pulse is delivered and the recorded signal from the instrumentation amplifier is processed.
9. The LabVIEW controller also automates a syringe pump (PHD Ultra, Harvard Apparatus, Holliston, Mass.) for cell suspension and sheath flow delivery, controls the electromechanical valves for outlet flow switching (Pneumadyne, Plymouth, Minn.), and maintains the temperature by modulating the current through the ITO heater using a closed-loop proportional-integral-derivative (PID) controller.

### Microfluidic Device Fabrication

The following is another description of a microfluidic device fabrication in accoradance with one embodiment. Glass slides (Fisher 12-550C) were cut to 50×50 mm using a handheld glass cutter and cleaned for 10 min in a Piranha bath at 120° C. (1:5 H₂O₂:H₂SO₄). Shipley S1818 photoresist (PR) was spun onto the slides at 4000 RPM for 35 s, leaving a ˜2 μm film. PR was soft baked for 5 min on a 90° C. hot plate. PR was then exposed on a contact mask aligner (Quintel Q4000) at 175 mJ/cm² (g-line) and subsequently developed in 1:1 MicroDev:H₂O for 35 s, rinsed with DI water and dried with N₂. The substrate was descumed in an O2 plasma device at 50 W for 1 min to improve metal adhesion. Then, 10 nm of Ti and 100 nm of Pt were evaporated in an e-beam evaporator, both at 0.1 nm/s (Edwards 306 E-Beam System). Film thickness was continuously monitored using a crystal monitor during deposition. Sheet resistance of metal film was measured at ˜4 Ω/square using a four-point resistivity probe. Liftoff was performed by sonicating substrates in acetone for 10 min using a fluoropolymer stand which kept them upright to avoid metal redeposition onto the glass. Remaining PR residue was wiped clean with an acetone soaked tissue, and slides were rinsed with isopropanol and DI water and then blown dry with N₂. Metal film was inspected for pinholes under transmission brightfield microscopy. Next, 400 nm of Si₃N₄ was deposited using plasma-enhanced chemical vapor deposition (PECVD) with 200 sccm NH₃, 200 sccm Ar, 40 sccm SiH₄, 25 W RF plasma, at 900 mTorr chamber pressure and 350° C. substrate temperature. (Oxford Instruments PlasmaLab 80 Plus). PR was again spin coated, patterned, developed, and descumed using the previous procedure to define the electrodes and contact pads. The Si₃N₄ was etched using SF₆ reactive ion etching (RIE) at 200 W for 4 min, using 15 sccm SF₆ and 5 sccm O₂, with a 290 mTorr chamber pressure (Reactive Ion Etching System, Plasma Equipment Technology Services). PR was stripped in acetone and the substrates were again cleaned with isopropanol and DI water. Single-layer SU8/silicon molds were prepared using established methods and subsequently treated with Trichloro(1H,1H,2H,2H-perfluorooctyl)silane (Sigma MKBC9893) vapor in a dessicator chamber for >2 hr to provide a non-stick coating. Polydimethylsiloxane (PDMS, Sylgard 184) was prepared with 1:10 w/w ratio of curing agent to prepolymer, thoroughly mixed, centrifuged to remove bubbles, and poured onto SU8/silicon molds at a thickness of ˜5mm. Following dessication to completely remove bubbles (generally 1-2 hr under house vacuum), the PDMS was oven cured at 60° C. for >2 hr and then peeled from the mold. Access holes were punched through the PDMS. Finally, the PDMS and electrode/glass substrate were simultaneously exposed to O₂ plasma at 100 W for 15 s to prepare the surfaces for covalent bonding. To align the PDMS to the electrodes, two small pieces of scotch tape were attached to the edges of the PDMS to provide a thin spacer, and the device was manually aligned to alignment marks on the substrate under a stereo scope. The PDMS was then pushed down, initiating bonding to the glass, and the tape was removed. The bonded devices were baked at 60° C. for >20 min. Detection electrodes were platinizated by flowing a solution of chloroplatinic acid (1.4% v/v) and lead acetate (0.02% w/v) in deionized (DI) water through the device and applying a −1.6V DC potential to each 20 μm electrode (vs. Pt reference) for 30 s.

Flow channel dimensions may be varied according to application. In one embodiment, the channel widths ranges from about 100-1000 microns, and channel heights ranges from about 50-1000 microns. In another embodiment, the channel width is about 1000 microns with a height of about 500 microns. In yet another embodiment, the channels width and height range from about 5-50 microns. In still another embodiment, the flow chamber is about 10 microns by about 10 microns.

### System Operation.

FIG. 2 depicts the operation of an exemplary system. Individual cells or cell clusters are introduced into the cell sorting system as a dilute suspension through a central channel and hydrodynamically focused over a detection region using flanking sheath flows. Two detection electrodes on the floor of the channel, one which is positioned directly under the cell and one which is positioned several cell radii away from the cell (transverse to the flow), measure the differential voltage signal generated by the cell using a low-noise instrumentation amplifier. When a cell passes into the detection region, it causes a drop in impedance between these two electrodes, in accordance with the Coulter principle. When this drop in impedance is detected, a short electrical pulse is delivered through two large stimulus electrodes positioned directly upstream and downstream of the detection electrodes. If longer recordings are desired (for example, to detect spontaneous beating or to examine the cell's response under multiple stimulus conditions), the flow can be stopped so that the cell is stationary. Due to their geometry in the channel, the stimulus and detection electrodes form a balanced bridge circuit, with the detection electrodes on an equipotential line in the stimulus field. The stimulus artifact seen by the amplifier is common-mode and thus rejected. Capacitive coupling of the stimulus and detection electrodes still leads to some artifact, which is removed in software. Based on automated analysis of the field potential, the outlet flow is switched to one of several output reservoirs using external electromechanical valves.

### Experimental Procedure

A study was conducted where iPSC-CM clusters, which were spontaneously contracting, were identified under a microscope and scraped from their culture well using a finely drawn sterile Pasteur pipette. These clusters were allowed to incubate for one hour, causing them to round up prior to experiments. Both iPSC-CM and undifferentiated iPSC clusters were drawn into a syringe, along with a small volume of culture medium. The syringe was connected to the inlet of the device and pushed either by hand or by using a syringe pump automated with the LabVIEW controller software. For cell detection experiments, cells were flown at a constant velocity while the electrode impedance was monitored continuously. For electrophysiology experiments, cells were positioned over the detection electrodes and the flow was stopped. Most iPSC-CM clusters visibly contracted spontaneously in the channel. All clusters contracted during stimulation. Cells could be repeatedly stimulated with no apparent degradation in signal strength or cell viability for over an hour.

### Experimental Results.

### Artifact Reduction.

Most extracellular electrophysiology is concerned with how signals propagate in 2D tissue preparations. Therefore, the requirements on artifact suppression are relaxed, because it's generally not necessary to measure signals from the same cell that is being directly stimulated, and due to propagation delay in the tissue, stimulation and field potential onset are temporally decoupled. Here, since the same cell is used for stimulation and recordation, careful consideration must be given to stimulus artifact suppression. There are three modes by which the stimulus signal can couple into the detection circuitry and introduce artifact: ohmic voltage gradients, common-mode conversion, and direct capacitive coupling between the stimulus and recording electrodes. To eliminate ohmic voltage gradients between the recording electrodes, a differential sensing scheme is employed where electrodes are placed on an equipotential line between the stimulus electrodes, essentially forming a balanced bridge circuit, as shown for example in FIG. 3. For the stimulus currents, voltage drops are well below the thermal noise floor. Common-mode conversion is mitigated by the use of a high-impedance instrumentation amplifier (10¹² Ω input impedance, 120 dB common mode rejection at 60 Hz), which does not share a common ground with the stimulator, preventing DC current from flowing from the stimulus electrodes into the recording amplifier. Capacitive coupling between the stimulus and recording leads is dramatically reduced by platinizing the electrodes, which increases the capacitance of the sensing electrodes from 110 pF to 13.9 nF. FIG. 4, for example, illustrates the effect these improvements had on reducing artifact. The remaining artifact for a typical 500 μs, 100 μA stimulus pulse was >1 mV. This is removed in software via template subtraction, whereby a template artifact signal uncontaminated by a field potential is subtracted from the signal, followed by least squares exponential curve fitting and subtraction of the remaining artifact.

### Enhanced Field Potentials of Cells in Micro-Channels

When adhered on conventional planar microelectrode arrays, iPSC-CMs produce field potentials around 100 μV. Cell adhesion is an important factor in obtaining good signal-to-noise ratios (SNR) in these recordings because cell adhesion may lead to a high resistance seal between the electrode and the extracellular medium. However, this seal is not necessary. The voltage drop in the vicinity of a cell is due to the diffusive ion flux through the membrane and the associated ionic current flowing radially around the cell. If one region of a cell membrane is presented with a much higher resistance to the bulk solution than the rest of the cell membrane (e.g., because it is adhered on a substrate), there will be less diffusive flux through that region, and the overall potential in the vicinity of the cell will not be substantially different than if the cell were unattached. On the other hand, if the cell is confined to a micro-channel with cross sectional area approaching that of the cell, the resistance increases nearly equally for the entire cell surface, and so the field potential amplitude in the cell vicinity will increase.

FIG. 5 shows an example of a field potential from iPSC-CM clusters adhered on an MEA along with signals from clusters confined to micro-channels of various sizes. As the cross sectional area of the channel decreases, the signal increases, thus allowing detection of field potentials from non-adhered cells. In one example, in which the cluster is confined to a tapered channel in which it is forced in contact with all 4 channels walls, the field potential amplitude is nearly double that of the attached MEA case.

Evoked and Spontaneous Signals Recorded from Undifferentiated iPSCs and iPSC-CMs

FIG. 6 shows spontaneous and evoked field potentials from an iPSC-CM cluster positioned over the detection electrode. This cluster produced spontaneous contractions at 5 Hz and was periodically stimulated at various frequencies and amplitudes (0.5 Hz, 100 μA pulses shown in this example) with no degradation in field potential amplitude for over one hour. In this example, the first two stimulus pulses occur during the refractory period from the last spontaneous contraction, so they did not result in evoked field potentials. The second two stimulus pulses do result in field potentials. Undifferentiated cells, on the other hand, produce no discernable signal over the noise floor, after the stimulus artifact is removed. When the flow is stopped and cells are stationary, multiple field potentials can be averaged to increase SNR by √N, provided that the field potential signal is consistent. This, of course, comes at the expense of throughput.

These results show the use of extracellular field potential recordings from suspended cells as a contrast signal for label-free cell sorting. When applied to neural or cardiovascular tissue engineering applications, this sorting technology promises a low false positive rate, because undifferentiated stem cells and most other differentiated cells do not express the voltage-gated ion channels required to produce a field potential signal. These results show that stimulus artifact can be completely eliminated to within 100 μs of the end of the stimulus pulse, and thus it is unlikely that the artifact would be mistaken for a field potential, which generally occurs >1 ms after stimulation. Although these signals are weaker than more traditional patch clamp signals, in which the transmembrane action potential is directly measured using an invasive pipette which breaks the cell membrane, the results indicate that they are nevertheless sufficient to distinguish differentiated and undifferentiated cell clusters. Clusters, rather than single cells, were chosen for experimentation because visible contraction is an easy way to confirm activity. Signals from clusters were observed as small as 70 μm in diameter. Single cell recordings may require careful attention to the dissociation procedure in order to preserve electrophysiological activity.

Unlike patch clamping, extracellular field potential recordings are completely non-invasive and preserve the viability of cells. The microelectrodes and the microfluidic channel can be used repeatedly for large numbers of cells, whereas pipettes used in patch clamping are generally discarded after each use. For these reasons, extracellular recordings are ideal for a sorting application.

### Throughput

Throughput may ultimately be limited by two factors: the duration of the field potential itself and the desired output purity. A cardiomyocyte field potential signal is approximately 100 ms in duration. Assuming there is exactly one cell in the channel at any instant and that analysis and switching requires negligible time, this sets an upper bound on throughput at about 10 cells/s per channel. Note that if only the depolarization spike (˜5 ms) is to be observed, the upper bound becomes 200 cells/s. However, as with fluorescence activated cell sorting (FACS), there is a tradeoff between throughput and output purity, since the probability of finding exactly one cell in the channel is governed by Poisson statistics (see supplementary information), and is always less than 1. Reducing the input sample concentration leads to fewer passenger cells (i.e., cells which happen to be in the channel while another cell is being analyzed, and which take the path of the analyzed cell). But lower sample concentrations also mean that for a larger portion of time, the device is idle. The presented impedimetric detection scheme places constraints on how fast cells can move through the device and still be detected. In the presented experiments, a 1 mm/s cell/cluster velocity was chosen, although this can be significantly increased with a higher sampling rate impedance analyzer. Assuming a minimum output purity of >95% is desired and a switching volume (that is, the volume between the interrogation region and the outlet channel) of 6 nL, an input sample concentration of <60,000 cells/mL would be required. This would also mean that for >70% of the time, there are no cells in the channel, according to Poisson statistics. So at a cell velocity of 1 mm/s, the maximum throughput drops to 0.3 cells/s. While this is quite low compared to modern FACS, it must be emphasized that an order of magnitude increase in cell throughput should be possible by simply improving cell detection speed.

There are two broad approaches to increasing throughput: parallelization and pipelining. Parallelization involves running multiple sorting channels simultaneously. Planar microfluidic devices are easily multiplexed, and as the detection methodology here is purely electrical and employs low-cost instrumentation, there is no limit to the number of parallel sorting channels that can be running simultaneously. A single device could easily carry 1000 independent sorting channels, and several examples of devices of this scale exist in the literature.

On-chip of off-chip pneumatic or electrostatic valving strategies can be integrated on a multiplexed chip to steer cells into a common set of outlets. Pipelining, on the other hand, would allow multiple cells in single file to be analyzed at once using an array of evenly-spaced electrodes which sample voltages at different regions along the channel. The field potential of a given cell would be reconstructed from these signals. Such an approach could allow for much higher flow rates, and so conventional FACS systems could be modified to include these electrode arrays.

### Stem Cell Culture

Induced pluripotent stem cells (iPSC) (iPS(IMR90) line, WiCell, Madison, Wis.) were maintained in the pluripotent state in 6-well tissue culture plates through daily feeding (2 mL/well) with mTeSR1 media (StemCell Technologies, Vancouver, Canada) supplemented with 1× penicillin/streptomycin (Invitrogen, #15140-163, Carlsbad, Calif.). Cells were passaged approximately every 4-6 days, at the time when colonies had expanded enough to begin merging with one another. Prior to passaging, new wells were coated with hESC/iPSC-qualified Matrigel (BD Biosciences, #354277, San Jose, Calif.) diluted in DMEM (Invitrogen, #10569, Carlsbad, Calif.) (75 microliters of Matrigel per 6 mL of DMEM, 1.0 mL of solution per well) and allowed to incubate at room temperature for at least one hour. Cells were removed from their plates mechanically using a scraping tool (Corning, #3008, Lowell, Mass.) while still in mTeSR1 from the previous day. The subsequently created cell-media mixture was triturated up and down approximately 5 times with a 5 mL pipette, and approximately 75-100 microliters of cell-media mixture were then transferred to each new well of a Matrigel pre-coated 6-well tissue culture plate. 2 mL of fresh mTeSR1 was subsequently added to each well, and the cells were allowed to incubate at 37° C. overnight to promote attachment. The remaining cells not transferred to a new plate were centrifuged at 300×g for 3 minutes, and then re-suspended in 90% Knockout Serum Replacement (KOSR) (Invitrogen, #10828010, Carlsbad, Calif.) with 10% DMSO (Sigma-Aldrich, #D2438, St. Louis, Mo.). 1 mL aliquots of cells in KOSR+DMSO were placed in cryovials and frozen at −80° C. overnight and then subsequently transferred to liquid nitrogen storage.

### Cardiomyocyte Differentiation

iPSC were cultured in 12-well tissue culture plates for differentiation. Prior to seeding cells on a plate, wells were coated with Matrigel (BD Biosciences, #354277, San Jose, Calif.) diluted in DMEM (Invitrogen, #10569, Carlsbad, Calif.) and allowed to incubate at room temperature for at least 1 hour. 75 microliters of Matrigel were diluted in 6 mL of DMEM, and 0.5 mL of the resulting solution was placed in each well. After at least one hour, the cells to be passaged were scraped off of their plate using a cell-scraping tool (Corning, #3008, Lowell, Mass.) while still in the mTeSR1 media from the previous day. The cell media suspension created was then triturated up and down approximately 5 times with a 5 mL pipette in order to break up the cell colonies. 25-50 microliters of cell-media suspension was then added to each of well of the Matrigel pre-coated 12-well plate. 1 mL of fresh mTeSR1 was then added to each well of the new plate, and the cells were allowed to incubate overnight to promote attachment. Differentiation was begun when the cells reached approximately 25-40% confluence, usually 2-4 days after initially seeding the cells. At this time, the cells were transferred to an RPMI (Invitrogen, #61870, Carlsbad, Calif.) media supplemented with B27 (Invitrogen, #17504-044, Carlsbad, Calif.), 1× non-essential amino acids (Invitrogen, #11140, Carlsbad, Calif.), 1× penicillin/streptomycin (Invitrogen, #15140-163, Carlsbad, Calif.), and 0.1 mM beta-mercaptoethanol (Invitrogen, #21985-023, Carlsbad, Calif.). On this first day (Day 0) of differentiation, 2 mL of RPMI media with 50 ng/mL of Activin A (R&D Systems, 338-AC, Minneapolis, Minn.) were added to each well On the subsequent day (Day 1) Activin A was removed, and 2 mL of RPMI media with 5 ng/mL of BMP-4 (R&D Systems, 314 BP, Minneapolis, Minn.) were added to each well. The cells were left in BMP-4 for approximately 48 hours. On Day 3, BMP-4 was removed, and 2 mL of fresh RPMI media was added to each well. RPMI media was subsequently replaced every 48 hours until Day 11, when the cells were transferred to a DMEM (Invitrogen, #10569, Carlsbad, Calif.) media supplemented with 5-10% FBS (Invitrogen, #10437028, Carlsbad, Calif.), 1× non-essential amino acids, 1× penicillin/streptomycin, and 0.1mM beta-mercaptoethanol. This DMEM media was then replaced (2 mL/well) approximately every 48 hours. Cardiomyocytes generally began spontaneously beating sometime between day 9 and day 20.

Undifferentiated iPSC Cluster Formation

To create clusters, undifferentiated iPSC cells were scraped from culture dishes and triturated as during normal passaging. The cell suspension was then transferred to a 12-well ultra-low-attachment culture plate at 100 uL per well. 1 mL of fresh mTeSR1 was then added to each well. Experiments with the clusters were carried out within 2 days.

### Poisson Statistics Governing Specificity and Throughput

The probability of finding exactly k cells in the sorting channel at any instant is governed by a Poisson distribution:

$$p ( k ) = \frac{{( CV )}^{k} {e}^{- CV}}{k !}$$

where C is cell concentration and V is switchable volume, that is, the volume between the electrode detection region and outlet channels which can be switched. For sorting small clusters, we have a switching volume of 60 μm wide×100 μm deep×1000 μm long=6 nL (note that for single cells, this volume would be smaller). As the cell suspension concentration increases, so does the probability of finding >1 cell in the switchable volume at any instant, as illustrated in the figure below. This may be the primary factor determining specificity, which implies that for a given cell suspension concentration, there is an upper bound on specificity, where

$$\begin{matrix}specificity = \frac{\# analyzed cells }{\# analyzed cells + \# passenger cells} \\ = p ( [ 0 , 1 ] ) \\ = {e}^{- CV} ( 1 + CV )\end{matrix}$$

However, the lower the cell concentration, the more time the device is spent idle, with no cells being interrogated. This will directly impact throughput.

Throughput is governed by two factors: the time required to analyze a cell and the mean time of arrival of cells in the chamber. The time required to analyze a cell or cluster may be fixed, limited by the duration of the field potential itself and the time required for processing. The field potential of a cardiomyocyte lasts about 100 ms following stimulation. Software processing and valve actuation requires about 100 ms. Therefore, a conservative estimate of total analysis time would be 300 ms per cell/cluster, setting the theoretical maximum throughput at 3.33 cells/s. The mean time of arrival of cells is determined by cell concentration and velocity. In our experiments, clusters can be reliably detected at a cell velocity, v, of about 1 min/s in a microchannel, where the cross-sectional area of the focused cell stream is A=60 μm×100 μm. This has not been optimized, and is primarily limited by the sampling rate of the impedance analyzer. The mean time between cell arrivals is:

Eₑᵣᵣ=1/vAC

Throughput is therefore the inverse of the sum of the analysis time and the mean arrival time:

$$throughput = \frac{1}{{\overline{t}}_{arr} + {t}_{anal}}$$

The above can be recast to find specificity versus throughput for different cell velocities (assuming a 300 ms analysis time).

$$C = {[ vA ( \frac{1}{throughput} - {l}_{anal} ) ]}^{- 1}$$

$$specificity = {e}^{- CV} ( 1 + CV )$$

Cell velocity of 1 mm/s, were used, which corresponds to a volumetric flow rate of 6.0 nL/s. At this velocity, a specificity of >95% implies a throughput of 0.3 cells/s. By improving impedimetric cell detection speed, higher cell velocities can be utilized. Throughputs >1 cells may be achieved.

### Stimulation of Single, Non-Adhered Cardiomyocytes in a Microfluidic Device

Towards the goal of single cell analysis, it was shown to be possible to repeatedly stimulate single HL1 cardiomyocytes and observe their depolarization using a calcium dye. HL1 cardiomyocytes were grown to 70% confluence in 25 mL flasks and then enzymatically dissociated in 1× Trypsin to obtain a single cell suspension. Single cells were manually trapped in a microfluidic device via light suction (leaving the membrane intact). The cell was stimulated with current pulses at 1 s intervals. Depolarization was observed using the Fluo-4 intracellular Ca²⁺ dye. Fluorescence intensity plot versus time were observed. Individual cells could be repeatedly stimulated for several minutes without fatigue.

### Multi-Electrode Array (MEA) Electrophysiology

Multi-electrode arrays (MEAs) with sixty 30 μm titanium nitride electrodes with indium tin oxide (ITO) contact traces equally spaced 200 μm apart and with an internal reference (Multi Channel Systems, MCS GmbH, Reutlingen, Germany, #Thin MEA 200/30 iR ITO) were sterilized through washing with 70% ethanol and placement under UV light for 30 minutes. MEAs were then washed with PBS (Invitrogen, Carlsbad, Calif., #10010) and plasma treated for 10 minutes. MEAs were then coated with 25 μg/mL fibronectin (Sigma-Aldrich, St. Louis, Mo., #F1141) and allowed to incubate at 37° C. for at least 30 minutes. Desired cardiomyocyte colonies were then manually dissected off their plates, transferred to the MEAs, and positioned on the electrodes using a flame-drawn glass pipette. The MEAs were placed in an incubated Zeiss Axio Observer Z1 microscope (Carl Zeiss, Gottingen, Germany) and the cardiomyocytes were allowed to incubate in approximately 800 μL of DMEM/10% FBS media for 12 hours at 37° C. to promote attachment.

A single MEA containing cells and DMEM/10% FBS or Tyrode's solution (Sigma, St. Louis, Mo., #T2397) was then placed in the amplifier (MCS, Reutlingen, Germany, #MEA 1060-Inv-BC) for recordings. The signals from the amplifier were sent to a SCB-68 shielded connector block (National Instruments (NI), Austin, Tex., #776844-01) and other data acquisition and control signals were routed through a BNC-2120 shielded connector block (NI, Austin, Tex., #777960-01). Signals from both connector blocks were then routed to a USB-6225 M Series DAQ (NI, Austin, Tex. #779974-01). Finally, signals acquired at 10,000 samples at 1 kHz from the DAQ were routed to a Dell Precision T3400 computer with a 2.40 GHz Intel Q6600 Quad Core Processer and 4 GB of RAM. Power to the MEA was provided through a PS2OW external power supply (MCS, Reutlingen, Germany).

Temperature (23-37° C.) at the MEA was sensed with a 100 Ohm Pt RTD element connected to a NI 9217 RTD analog input module (NI, Austin, Tex., 779592-01) within a NI Compact RIO-9024 Real-Time power PC embedded controller (NI, Austin, Tex., #781174-01). Heating was controlled via an analog output signal from the USB 6225 DAQ to a custom heating box delivering modulated electrical current to a resistive heater on the MEA amplifier. A gas mixture of humidified 95% air/5% CO₂ was constantly delivered to the cardiomyocytes within the MEA via a custom made incubation cover.

The MEA amplifier was configured with MEA Select 1.1.0 software (MCS, Reutlingen, Germany) and electrical, video, temperature, and gas signals were acquired and controlled with a custom program created with LabVIEW 8.6 (NI, Austin, Tex.).

### Electrophysiology as an Indicator of Stem Cell Differentiation and Maturity

Electrophysiology is the gold standard for subtyping neurons and cardiomyocytes, with different cell types producing dramatically different signals. Neurons, for example, are characterized by rapid Na+/K+ depolarization/repolarization currents and produce sharp field potential “spikes”. The refractory period for neurons is <10 ms. Cardiomyocytes, on the other hand, have relatively slow repolarization currents which may be accompanied by an additional Ca²⁺ inward current which causes the cell membrane to remain depolarized longer. This prolongs the field potential duration to about 100 ms, with refractory periods over 100 ms. Certain cardiomyocytes also undergo spontaneous depolarization (i.e. nodal pacemaker cells), and this too can be quantitatively assessed in our device. The heart is a mosaic of different myocyte phenotypes, including atrial and ventricular cardiomyocytes, nodal pacemaker cells, and vascular smooth muscle cells. Each of these cells has distinct electrophysiological properties. During development, the heart undergoes extensive remodeling, and so the electrophysiology of cardiomyocytes and smooth muscle is also an indicator of maturity. Field potential rise time, duration, and frequency of spontaneous contraction have all been shown to correlate with ES-derived cardiomyocyte maturation from an embryonic to an adult phenotype. Cardiomyocyte maturity is thought to be critical for tissue engineering applications, and it has been shown that within a given stem cell derived population, cardiomyocyte maturity is heterogeneous and does not necessarily correlate with age in culture.

Therefore, ex-vivo maturation may not be sufficient to produce suitable populations, and technologies which can sort cells based on maturity will be advantageous. FIG. 6 (panel C) illustrates the features of the field potential. The durations of the various phases of the cardiac action potential: depolarization (t_{dp}), plateau (t_{slow}), and repolarization (tᵣₚ) are particularly important when assessing phenotype, as well as whether or not the cell spontaneously beats, and if so, its intrinsic spike interval (tᵢₛᵢ).

Stem cells give rise to cardiomyocytes with action potential waveforms characteristic of nodal, atrial, and ventricular tissues. Although ventricular-like cardiomyocytes are desirable for most tissue engineering applications, there is currently no way to specifically isolate this fraction. Most stem cell differentiation protocols involve the production of cell clusters (such as embryoid bodies), and it has been shown that within a given cluster, a particular action potential type was dominant. Therefore, even sorting intact clusters (rather than individual cells) would be very useful.

In the presented experiments, differentiated iPSC outgrowths form clusters of cardiomyocytes, some of which have pacemaker-like activity and spontaneously contract at a frequency of 1-5 Hz. The spontaneous contraction frequency seems to depend primarily on differentiation, culture conditions, and temperature and is very consistent across a batch of cells and throughout the duration of an experiment. Other clusters in the same iPSC cultures do not spontaneously contract, but they do contract when stimulated. In a cardiac tissue engineering application, it is more desirable to implant cells which do not have pacemaker-like activity because they can lead to ectopic arrhythmias. The device proposed here can be used to isolate non-pacemaker-like clusters, making it well-suited for cardiac tissue engineering.

A cardiomyocyte's electrophysiological phenotype is intimately tied to the task which it must perform once implanted in the host organ, namely: produce an organized contraction in response to electrical excitation. We hypothesize that electrophysiological homogeneity of implanted cardiomyocytes will lead to improved systolic output, improved electromechanical coupling within the host myocardium, reduced incidence of arrhythmias, and improved graft viability. Electrophysiological sorting may substantially reduce the possibility of teratoma formation, because it is unlikely that undifferentiated cells will produce signals which could be mistaken as depolarization currents. This technology would also be useful in quantitatively assessing the effects of pharmacological agents on cardiomyocyte populations, which is an important requirement for drug toxicity screening. Finally, aside from its clinical applications, exploring the heterogeneity of electrophysiological phenotypes of cell populations derived from stem cells or progenitors would provide insight into fundamental questions in developmental and stem cell biology.

### Computer Implementation.

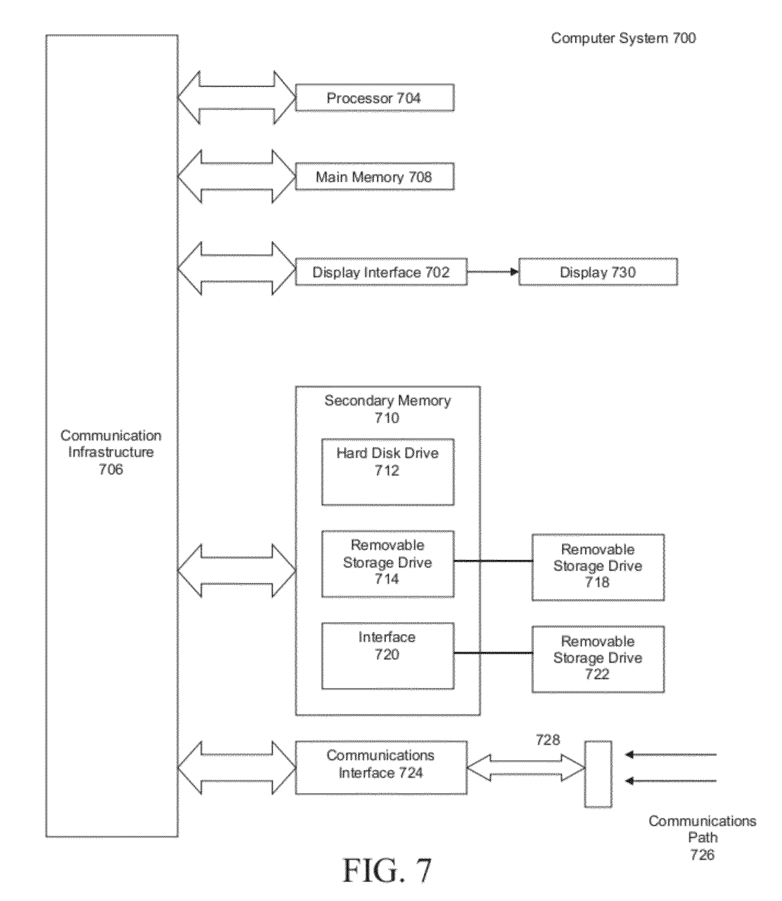

FIG. 7 is a schematic drawing of a computer system used to implement the methods presented herein. In one embodiment, the invention is directed toward one or more computer systems capable of carrying out the functionality described herein. An example of a computer system 700 is shown in FIG. 7. Computer system 700 includes one or more processors, such as processor 704. The processor 704 is connected to a communication infrastructure 706 (e.g., a communications bus, cross-over bar, or network). Computer system 700 can include a display interface 702 that forwards graphics, text, and other data from the communication infrastructure 706 (or from a frame buffer not shown) for display on a local or remote display unit 730.

Computer system 700 also includes a main memory 708, such as random access memory (RAM), and may also include a secondary memory 710. The secondary memory 710 may include, for example, a hard disk drive 712 and/or a removable storage drive 714, representing a floppy disk drive, a magnetic tape drive, an optical disk drive, flash memory device, etc. The removable storage drive 714 reads from and/or writes to a removable storage unit 718 in a well known manner. Removable storage unit 718 represents a floppy disk, magnetic tape, optical disk, flash memory device, etc., which is read by and written to by removable storage drive 714. As will be appreciated, the removable storage unit 718 includes a computer usable storage medium having stored therein computer software and/or data.

In alternative embodiments, secondary memory 710 may include other similar devices for allowing computer programs or other instructions to be loaded into computer system 700. Such devices may include, for example, a removable storage unit 722 and an interface 720. Examples of such may include 2 program cartridge and cartridge interface (such as that found in video game devices), a removable memory chip (such as an erasable programmable read only memory (EPROM), or programmable read only memory (PROM)) and associated socket, and other removable storage units 722 and interfaces 720, which allow software and data to be transferred from the removable storage unit 722 to computer system 700.

Computer system 700 may also include a communications interface 724. Communications interface 724 allows software and data to be transferred between computer system 700 and external devices. Examples of communications interface 724 may include a modem, a network interface (such as an Ethernet card), a communications port, a Personal Computer Memory Card International Association (PCMCIA) slot and card, etc. Software and data transferred via communications interface 724 are in the form of signals 728 which may be electronic, electromagnetic, optical or other signals capable of being received by communications interface 724. These signals 728 are provided to communications interface 724 via a communications path (e.g., channel) 726. This channel 726 carries signals 728 and may be implemented using wire or cable, fiber optics, a telephone line, a cellular link, a radio frequency (RF) link, a wireless communication link, and other communications channels.

In this document, the terms “computer-readable storage medium,” “computer program medium,” and “computer usable medium” are used to generally refer to media such as removable storage drive 714, removable storage units 718, 722, data transmitted via communications interface 724, and/or a hard disk installed in hard disk drive 712. These computer program products provide software to computer system 700. Embodiments of the present invention are directed to such computer program products.

Computer programs (also referred to as computer control logic) are stored in main memory 708 and/or secondary memory 710. Computer programs may also be received via communications interface 724. Such computer programs, when executed, enable the computer system 700 to perform the features of the present invention, as discussed herein. In particular, the computer programs, when executed, enable the processor 704 to perform the features of the presented methods. Accordingly, such computer programs represent controllers of the computer system 700. Where appropriate, the processor 704, associated components, and equivalent systems and sub-systems thus serve as “means for” performing selected operations and functions.

In an embodiment where the invention is implemented using software, the software may be stored in a computer program product and loaded into computer system 700 using removable storage drive 714, interface 720, hard drive 712, or communications interface 724. The control logic (software), when executed by the processor 704, causes the processor 704 to perform the functions and methods described herein.

In another embodiment, the methods are implemented primarily in hardware using, for example, hardware components such as application specific integrated circuits (ASICs). Implementation of the hardware state machine so as to perform the functions and methods described herein will be apparent to persons skilled in the relevant art(s). In yet another embodiment, the methods are implemented using a combination of both hardware and software.

Embodiments of the invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing firmware, software, routines, instructions, etc.

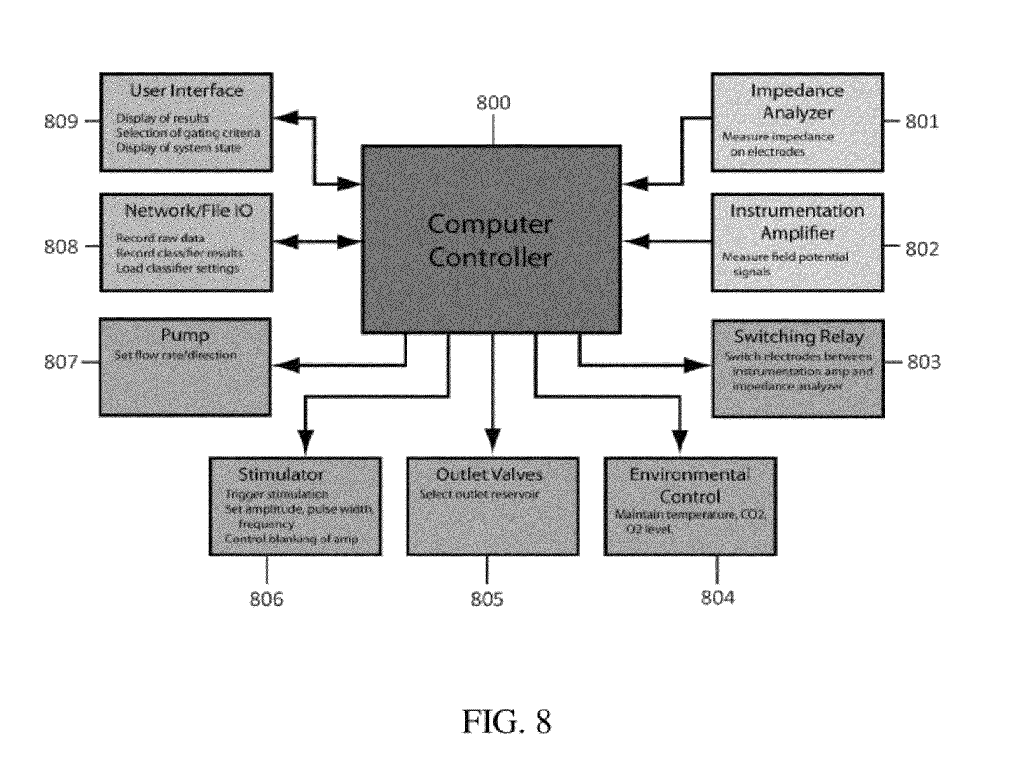

FIG. 8 illustrates components of an automated, specialized computer-controlled cell sorter system. More specifically, FIG. 8 illustrates the organization of the computer controller 800 and the various components of the cell sorter/cytometer system that it automates. The computer controller 800, for example of FIG. 8, receives input from the impedance analyzer 801 and recording amplifier 802 and controls the switching relay 803, environmental control 804, outlet valves 805, stimulator 806, and cell delivery pump 807. Raw data may be recorded to a disk or network location 808 for later analysis. The user may interact with the system through a graphical or text-based user interface 809 to observe the sorting/cytometry analysis results. The computer controller may, for example, utilize the Labview software development environment. The computer is responsible for controlling the pump 807 which delivers the cells into the detection channel (syringe pump, pressure controller, etc.). It may, for example, control the pump via a USB or RS232 serial interface.

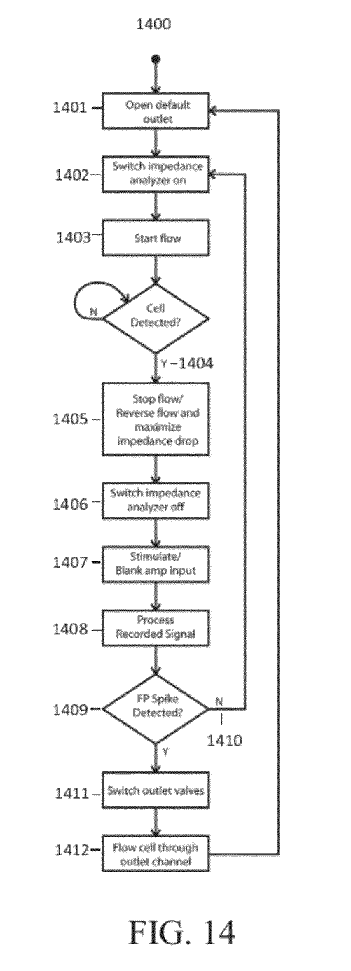

FIG. 14 shows a representative software state diagram for cell sorting. More specifically, FIG. 14 illustrates the software algorithm for detecting and analyzing cells for sorting 1400. The software algorithm 1400 begins by opening a default outlet 1401. A “default” outlet valve is selected to ensure that any unwanted debris is sent to a waste outlet. The impedance analyzer is then switched on 1402 and the flow is started 1403 through the device. As the pump is pushing fluid through the device, the impedance on the detection electrodes is constantly being monitored (separate, upstream detection electrodes could also be used). Impedance may be monitored using a lock-in amplifier, dedicated network analyzer IC, or a commercial LCR meter. A typical interrogation frequency for cell detection is 100 kHz. Typical impedance values with microelectrodes will be in the range of 10-100 kohms, and the presence of a cell may increase this value by as much as 20%.

When an increase in impedance is detected above a certain threshold, the computer interprets this as a cell passage 1404. Depending on the type of analysis, the pump may be stopped during analysis or may continue during analysis. The flow is optionally stopped if cell passage is detected 1405. The impedance analyzer is turned off 1406 and/or disconnected from the flow channel to avoid interference using, for example, relay switches. One or more stimulus pulse(s) 1407 are delivered to the cells through dedicated stimulus electrodes. The stimulus pulses may be generated in a digital buffer on the computer and delivered through a digital to analog converter or a commercial data acquisition module (DAQ). The stimulus pulse is delivered using a stimulus circuit 806 which is isolated from the recording amplifier 802. The voltage signal on the microelectrode near the cells is simultaneously recorded 1408 through an instrumentation amplifier with a typical gain of 1000. Typical sampling rates for this signal are in the range of 1-100 kHz, and a typical range for this signal is +/−1V (after amplification). The recorded signal is analyzed 1409, and the contaminating stimulus artifacts is/are removed. The resulting evoked field potential(s) and/or spontaneous field potentials from the cells are analyzed using a variety of possible algorithms (wavelet analysis, Fourier Transforms, thresholding, etc.). Typical analysis will focus on the amplitudes and durations of the various phases of the field potential (depolarization, contraction, and repolarization), as well as the spontaneous contraction frequency. If no field potential spike or corresponding measure is detected at this point, the impedance analyzer is switched on 1410.

Analysis may also include the response of the cells to different kinds of stimuli (where the frequency or amplitude may be swept, for example). Based on this analysis and the gating parameters that have been established in the software, a decision is made regarding the cell type. Outlet valves are switched 1411 to allow the cell to flow out of the analysis channel 1412 into the appropriate outlet reservoir. Outlet valves may be on the micro-device itself or may be external to it. The pump 807 is re-engaged to allow the cells to exit the channel, the default outlet valve is again switched open 1401, and the process 1400 is repeated for subsequent cells.

### Additional Embodiments

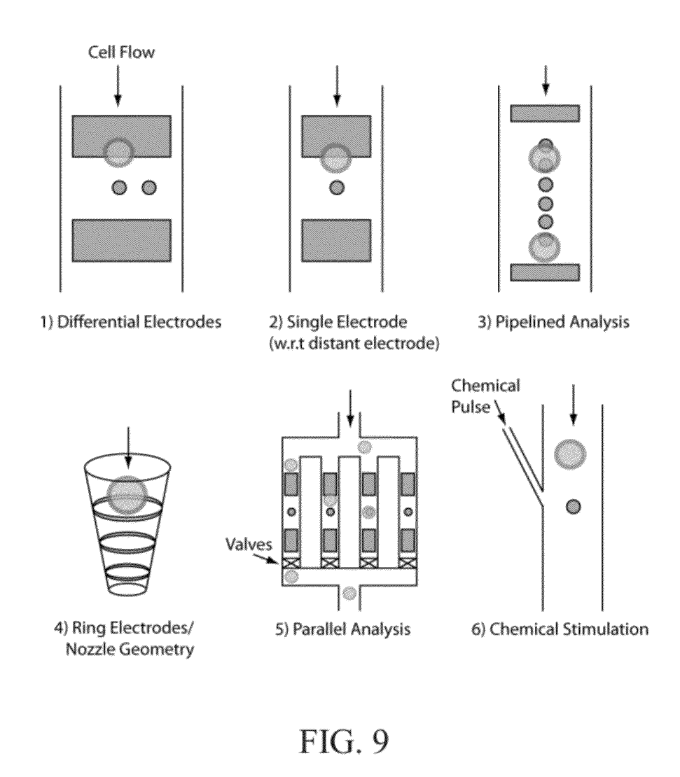

FIG. 9 shows various embodiments of electrophysiological cell sorting. In (1) differential stimulus and differential detection electrodes are positioned orthogonally to each other to minimize stimulus artifact. In (2) single detection electrode (reference electrode is placed elsewhere in the system). In (3) multiple electrodes are utilized to measure multiple field potentials from a single cell or to measure signals from multiple cells simultaneously (i.e. pipelining), which is one method of increasing throughput. In (4) a nozzle geometry is shown, utilizing ring-shaped electrodes within the wall of the nozzle. This configuration may be used in conjunction with conventional FACS/flow cytometer systems. In (5) parallel sorting channels allow analysis of multiple cells at once. Optionally, independently-addressable valves at each parallel channel allow them to be sorted independently. In (6) rather than an electrical current, a chemical pulse could be delivered through a side channel. Chemical pulses can also be used to elicit electrophysiological responses. Chemical pulses could include salt buffers, cytokines, proteins, or a fluid of a different temperature.

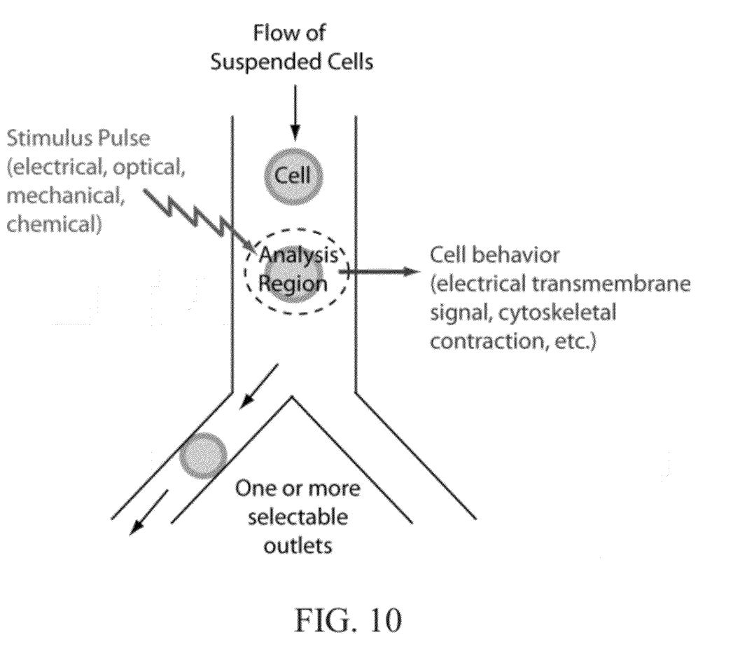

FIG. 10 shows cell sorting based on a generalized physiological response to stimulus. Stimulus may be electrical current/voltage pulses, optical pulses, mechanical (pressure, shear force) pulses, or chemical pulses. Cell behavior may be any physiological response of the cell which is produced as a result of the stimulus or independent of stimulation. This behavior could be measured through a variety of means. For example, transmembrane electrical currents can be measured using extracellular electrodes, transmembrane electrodes, voltage-sensitive dyes, or ion-sensitive dyes. Additionally, cytoskeletal contractions could be measured using video, laser scattering, or pressure transducers.

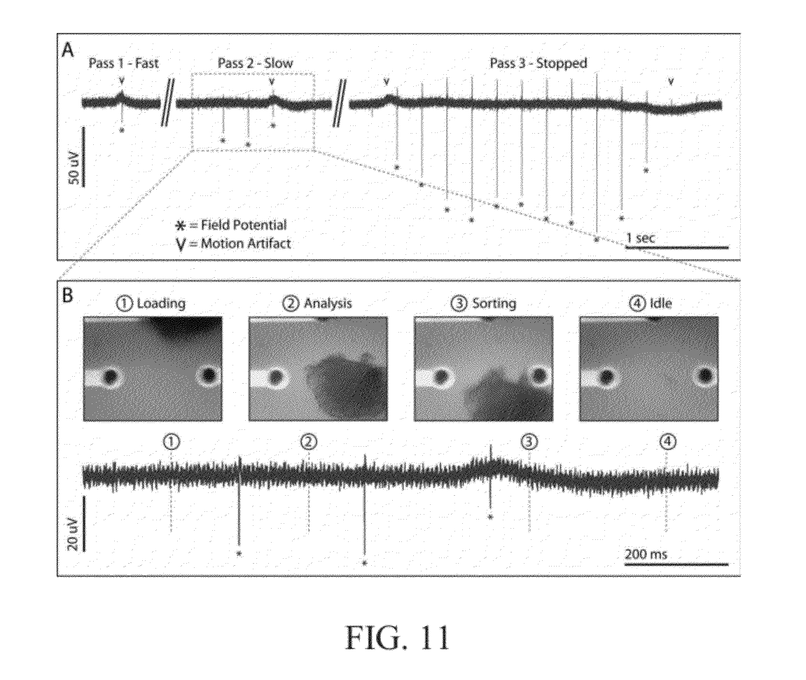

FIG. 11 shows spontaneous field potentials recorded from cells in flow at different flow rates.

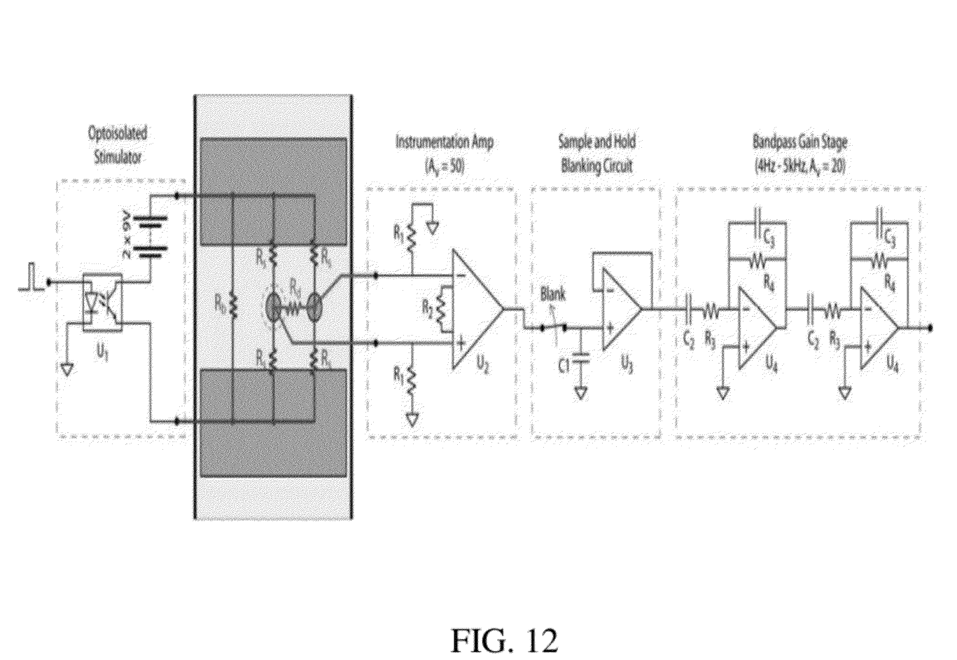

FIG. 12 shows An example of a stimulator and instrumentation amplifier developed for electrophysiological cell sorting.

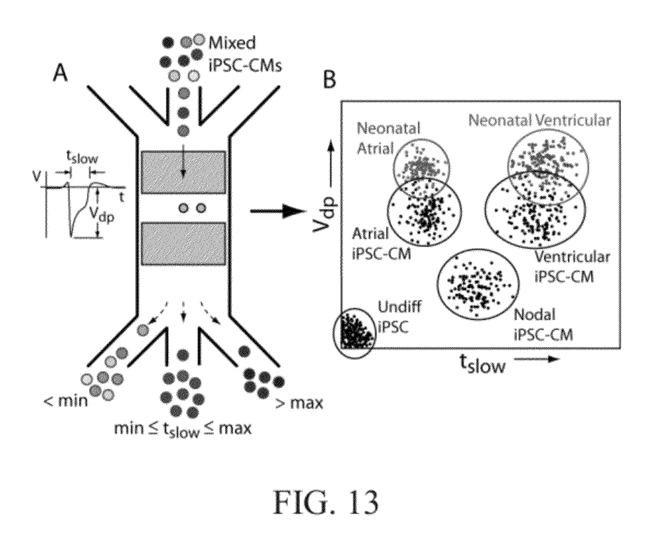

FIG. 13 shows example of field potential characteristics that may be used to assess cell phenotype. In the scatter plot, depolarization amplitude (Vdp) and contraction duration (tslow) are plotted, and different populations of cells cluster in different locations on this plot. The circles indicate gating regions that could be used to sort these cells.

### Conclusion

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. For example, although the present invention is particularly advantageous because it allows for non-genetic, label-free cell purification, the invention is not limited to use in non-genetic, label free cell sorting applications (unless otherwise claimed). Other uses and applications fall within the scope of the present invention.

It is intended that the appended claims be construed to include other alternative embodiments of the invention; including equivalent structures, components, methods, and means. It is to be appreciated that the Detailed Description section, and not the Summary and Abstract sections, is intended to be used to interpret the claims. The Summary and Abstract sections may set forth one or more, but not all exemplary embodiments of the present invention as contemplated by the inventor(s), and thus, are not intended to limit the present invention and the appended claims in any way.

What is claimed is:

1. A method, comprising:
flowing a cell population through a flow channel;
subjecting one or more individual cells to an electrical stimulus within the flow channel;
sensing an electrical response evoked by the stimulated cell;
obtaining an electrophysiological signature of the stimulated cell based on the evoked electrical response; and
sorting the stimulated cell based on its electrophysiological signature.

2. The method of claim 1, further comprising:
hydrodynamically focusing the cell population within the flow channel.

3. The method of claim 1, further comprising:
identifying a phenotype of the stimulated cell based on its electrophysiological signature.

4. The method of claim 1, further comprising:
identifying the stimulated cell's developmental maturity based on its electrophysiological signature.

5. The method of claim 1, further comprising:
evaluating the stimulated cell's cellular function based on its electrophysiological signature.

6. The method of claim 1, further comprising:
preparing the cell population by enzymatically digesting the cell population into a single cell suspension.

7. The method of claim 1, further comprising:
preparing the cell population by adhering the cell population onto or within a carrier.

8. The method of claim 7, wherein the carrier is a micro-scale polystyrene bead.

9. The method of claim 1, further comprising:
preparing the cell population by aggregating the cell population into a cluster.

10. The method of claim 1, wherein the stimulated cell is selected from the group consisting of: cardiomyocytes, neurons, smooth muscle cells, and pancreatic beta cells.

11. The method of claim 1, wherein the cell population is free of any cellular labeling.

12. The method of claim 1, wherein the cell population is free of any genetic modification.

13. A method, comprising:
stimulating a cell with a stimulus;
sensing a response evoked by the cell based on the stimulus;
identifying a phenotype of the cell based on the evoked response; and
sorting the cell based on its phenotype.

14. The method of claim 13, wherein the stimulation step further comprises:
stimulating the cell with a stimulus selected from the group consisting of: an electrical stimulus, a mechanical stimulus, an optical stimulus, a thermal stimulus, a chemical stimulus, and any combination thereof.

15. The method of claim 13, further comprising:
applying an electrical current pulse to the cell; and
sensing an extracellular electrophysiological field potential signal evoked from the cell in response to the applied electrical current pulse.

16. The method of claim 15, further comprising:
quantifying a parameter of the electrophysiological field potential signal, wherein the parameter is selected from the group consisting of: an amplitude and duration of depolarization, a sustained contraction phase, a repolarization phase, and any combination thereof.

17. A system, comprising:
a flow chamber having a cell inlet;
an impedance analyzer coupled to the flow cell and configured to detect when a cell has entered the flow chamber;
a stimulus pulse generator having two stimulation electrodes configured to create an electrical field across the flow chamber;
a signal detector having two sensing electrodes located on an equipotential line between the stimulation electrodes, wherein the two sensing electrodes are coupled to a differential sensing amp configured to detect an extracellular electrophysiological field potential signal evoked from the cell in response to the electrical field across the flow chamber;
a processing unit coupled to the signal detector and configured to identify a phenotype of a cell in the flow chamber based on the detected electrophysiological field potential signal evoked from the cell;
a cell collection chamber coupled to the flow chamber and configured to receive a cell of interest based on the cell's phenotype; and
a drain outlet coupled to the flow and configured to receive unwanted cells or fluid from the flow chamber.

18. The system of claim 17, wherein the cell of interest is selected from the group consisting of: cardiomyocytes, neurons, smooth muscle cells, and pancreatic beta cells.

19. The system of claim 17, wherein the processing unit is configured to identifying the cell's developmental maturity.

20. The system of claim 17, wherein the processing unit is configured to evaluate the cell's cellular function.

21. The method of claim 7, wherein the carrier is a micro-scale polymer matrix within which the cell(s) can infiltrate.

22. A method, comprising:
flowing a cell population through a flow channel;
sensing a spontaneous electrical response from a cell; and obtaining an electrophysiological signature of the cell based on the electrical response.

23. The method of claim 22, further comprising:
sorting the cell based on its electrophysiological signature.

24. A method, comprising:
sensing a spontaneous electrical response from a cell; and
identifying a phenotype of the cell based on the electrical response.

25. The method of claim 24, further comprising:
sorting the cell based on its phenotype.
